(19) United States
(12) Patent Application Publication
(10) Pub. No.: US 2024/0008569 A1
(43) Pub. Date: Jan. 11, 2024

(54) Face Mask Providing For Scent Releasing Layer

(71) Applicants: Charles Alvah Hill, Longmont, CO (US); Christopher Gust Papas, Longmont, CO (US)

(72) Inventors: Charles Alvah Hill, Longmont, CO (US); Christopher Gust Papas, Longmont, CO (US)

(21) Appl. No.: 18/251,677

(22) PCT Filed: Nov. 2, 2021
(86) PCT No.: PCT/US2021/057763

§ 371 Date: May 3, 2023

Related U.S. Application Data
(60) Provisional application No. 63/109,681, filed on Nov. 4, 2020.

(51) Int. Cl. A41D 13/11; A61M 21/00; A61L 9/12

(52) U.S. Cl. CPC A41D 13/1192; A41D 13/113; A41D 13/1161; A61M 21/00; A61L 9/127; A41D 2400/36; A61M 2021/0016

(57) ABSTRACT

Face masks are described which provide for a scent releasing layer. In certain aspects, cloth face masks are described having a pocket into which a scent releasing layer may be placed. In certain other aspects, disposable facemasks are described which contain a scent releasing layer within the inner and outer layer of the face mask.

### CROSS-REFERENCE TO RELATED APPLICATIONS

This patent application claims the benefit of priority to co-pending U.S. Provisional Patent Application No. 63/109,681 filed Nov. 4, 2020, which is hereby incorporated by reference in its entirety.

### BACKGROUND OF INVENTION

Prior to the COVID-19 pandemic, face masks were worn primarily by hospital workers or cleaning workers to protect against illnesses caused by various types of microorganisms. However, the COVID-19 pandemic has caused the use of face masks to become widespread. Not only is mask usage more common day-to-day for a larger portion of the public, but the amount of time for which people wear masks has also substantially increased.

While the wearing of masks provides certain health benefits, the experience of wearing a mask has certain drawbacks which can include the experiencing of more unpleasant smells, which, in some instances, may originate from the wearer of the mask. Thus, in the context of more frequent usage of face masks by a larger portion of the public for longer periods of time, there is a need in the market for face masks which can provide for the release of scents desirable to the wearer.

### SUMMARY OF THE INVENTION

The present disclosure provides, in various aspects, face masks with a scent releasing layer. In one aspect, the present disclosure provides a cloth mask with a pocket having an opening into which a scent releasing layer may be placed. In another aspect, the present disclosure provides a disposable mask containing a scent releasing layer between the front (outer layer) and the inner (facial contact) layer.

In certain aspects, a cloth mask is disclosed herein comprising an inner facial contact surface and an outer surface, wherein the inner facial contact surface and the outer surface are configured to cover the nose and mouth of a user, wherein the mask comprises at least one strap to support the mask on the face of the user, further wherein either the inner facial contact surface or the outer surface of the mask comprises a pocket which is smaller in surface area than either the inner facial contact surface or the outer surface, wherein the pocket is attached to the inner or outer surface of the mask on all but one side, wherein the unattached side of the pocket provides an opening allowing access to a cavity within the pocket.

In certain further aspects, a disposable mask is disclosed herein comprising: an inner facial contact layer, a middle layer and an outer layer, wherein the inner facial contact layer, middle layer and outer layer are each configured to cover the nose and mouth of a user, wherein the mask comprises at least one strap to support the mask on the face of the user, further wherein the mask comprises a scent releasing layer positioned between the inner facial contact layer and the outer layer.

The following numbered paragraphs [008]-[031] contain statements of broad combinations of the inventive technical features herein disclosed:

1. A disposable face mask, comprising: an inner facial contact layer, a middle layer and an outer layer, wherein the inner facial contact layer, middle layer and outer layer are each configured to cover the nose and mouth of a user, wherein the mask comprises at least one strap to support the mask on the face of the user, further wherein the mask comprises a scent releasing layer positioned between the inner facial contact layer and the outer layer.

2. The disposable face mask of aspect 1, wherein the middle layer provides for filtering of air inhaled and/or exhaled by the user.

3. The disposable face mask of aspect 1 or 2, wherein the scent releasing layer is positioned between the middle layer and the inner facial contact layer.

4. The disposable face mask of aspect 1 or 2, wherein the scent releasing layer is positioned between the middle layer and the outer layer.

5. The disposable face mask of any one of aspects 1-4, wherein the scent releasing layer comprises a water soluble polymer selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

6. The disposable face mask of aspect 5, wherein the water soluble polymer is pullulan.

7. The disposable face mask of any one of aspects 1-6, wherein the scent releasing layer comprises essential oils.

8. The disposable face mask of any one of aspects 1-7, wherein the scent releasing layer comprises a compound selected from the group consisting of thymol, methyl salicylate, eucalyptol, menthol and any combination thereof.

9. The disposable face mask of any one of aspects 1-8, wherein the facial contact layer, middle layer and outer layer each comprises a melt-blown polymer.

10. The disposable face mask of aspect 9, wherein the melt-blown polymer is polypropylene.

11. The disposable face mask of aspect 3, wherein the scent releasing layer is layered on the proximal surface of the middle layer.

12. The disposable face mask of aspect 4, wherein the scent releasing layer is layered on the distal surface of the middle layer.

13. The disposable face mask of any one of aspects 1-12, wherein the scent of the scent releasing layer is designed to expire after a predetermined period of time to indicate that the useful life of the mask has expired.

14. A reusable cloth face mask, comprising an inner facial contact surface and an outer surface, wherein the inner facial contact surface and the outer surface are configured to cover the nose and mouth of a user, wherein the mask comprises at least one strap to support the mask on the face of the user, further wherein either the inner facial contact surface or the outer surface of the mask comprises a pocket which is smaller in surface area than either the inner facial contact surface or the outer surface, wherein the pocket is attached to the inner or outer surface of the mask on all but one side, wherein the unattached side of the pocket provides an opening allowing access to a cavity within the pocket.

15. The reusable cloth face mask of aspect 14, wherein the pocket is stitched on to the inner facial contact surface.

16. The reusable cloth face mask of aspect 14, wherein the pocket is stitched on to the outer surface.

17. The reusable cloth face mask of any one of aspects 14-16, wherein the pocket is in the shape of a quadrilateral.

18. The reusable cloth face mask of any one of aspects 14-17, wherein the mask comprises a central vertical stitched seam, wherein one side of the pocket is positioned such that the vertical seam sews the one side of the pocket to the inner or outer surface of the mask.

19. The reusable cloth face mask of any one of aspects 14-18, wherein the opening of the pocket is located opposite the side of the pocket sewn into the central vertical seam.

20. The reusable cloth face mask of any one of aspects 14-19, wherein the pocket is made of cloth.

21. The reusable cloth face mask of any one of aspects 14-20, wherein the inner facial surface and/or the outer surface comprises an antimicrobial compound.

22. The reusable cloth face mask of any one of aspects 14-21, wherein the face mask is packaged in the same packaging as a scent releasing layer which is sized to fit in the pocket.

23. The reusable cloth face mask of any one of aspects 14-22, wherein the pocket further comprises a means for closing access to the cavity within the pocket.

24. A method of making a disposable mask which indicates an observable expiration of useful life by:
(1) making a disposable face mask, comprising: an inner facial contact layer, a middle layer and an outer layer, wherein the inner facial contact layer, middle layer and outer layer are each configured to cover the nose and mouth of a user, wherein the mask comprises at least one strap to support the mask on the face of the user, further wherein the mask comprises a scent releasing layer positioned between the inner facial contact layer and the outer layer; and
(2) designing the scent releasing layer such that the scent of the scent releasing layer expires after a predetermined period of time to indicate to the user that the useful life of the mask has expired.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a diagram of a reusable cloth mask with a pocket for receiving a scent releasing layer. FIG. 1 shows a scent releasing layer (1) (hashed lines enclosed by gray dashed lines) inside of pocket (2) (grey rectangular border within black dashed lines on bottom, top and left) sewn on 3 sides (black dashed lines) to reusable mask with an open side of the pocket (gray border outside of gray dashed lines but not enclosed by black dashed lines) as indicated by the arrow (3).

FIG. 2 shows an exploded view from back of a disposable mask having an internal scent releasing layer showing 3 layers for covering the nose and mouth of a wearer including a front layer (1), a middle layer (2), an inner facial contact layer (3). The view further shows a scent releasing layer (4) between the front layer (1) and the inner facial contact layer (3).

FIG. 3 shows a side view of a disposable mask having an internal scent releasing layer showing the 3 layers for covering the nose and mouth of a wearer including a front layer (1), a middle layer (2), an inner facial contact layer (3). The side view further shows a scent releasing layer (4) between the front layer (1) and the inner facial contact layer (3). While the side view shows a scent releasing layer (4) on both sides of the middle layer, in some aspects, the scent releasing layer (4) appears in only one of the two illustrated positions.

### DETAILED DESCRIPTION OF THE INVENTION

As used herein, the term “cloth” refers to any material which may be washed and reused. In certain aspects, cloth may refer to a woven or felted fabric made of natural fibers (e.g. cotton), synthetic fibers (e.g. polyester), or any combination thereof.

As used herein, the term “disposable” refers to an intention on the part of the manufactured that the relevant article (e.g. a mask) be used only for a single use. In certain aspects, the use of the article is intended to be for a limited time.

As used herein, the term “expire” or expired” refers to a coming to an end, not necessarily an absolute end. Thus, expired can refer to a subjective end. In certain aspects, expired refers to the scent of a scent releasing layer, in which case the end of the scent refers to a substantial diminishment in the strength of the released scent to the point where an observer, optionally the wearer of the mask, considers the scent to have expired.

As used herein, the terms “filter” or“filtering” refer to removal of undesirable particles (e.g. microorganisms) present in the air.

As used herein, the term “mask” refers to an object normally worn on the face of a wearer. In certain preferred aspects, a mask is designed to cover the nose and mouth of a user.

As used herein, the term “melt-blown” refers to a material produced by the conventional fabrication method where a polymer melt is extruded through small nozzles surrounded by high speed blowing gas.

As used herein, the term “pocket” refers to a bag or envelop-like receptacle which is fastened to the surface of or inserted with the body of a mask.

As used herein. “scent releasing” refers to emission of an observable scent which may be considered desirable by some observers.

As used herein “useful life” refers to an intention on the part of the manufactured that the relevant article should be used only for predetermined duration of time. In certain aspects, the predetermined duration of the useful life of the mask is determined based on the length of time for which the scent releasing layer releases scent.

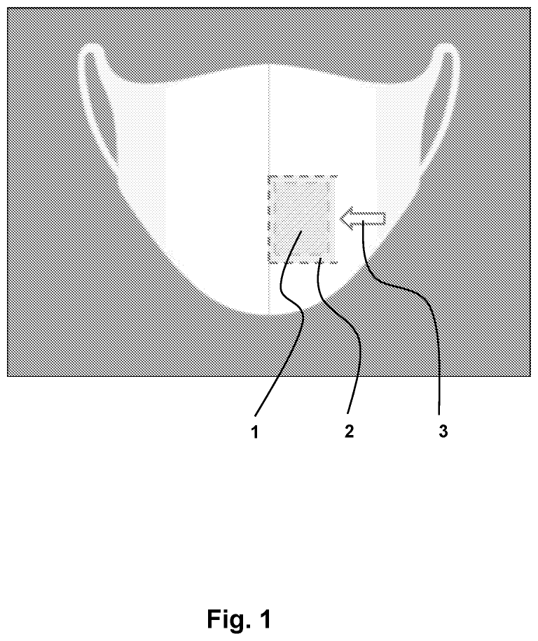

In one aspect, a reusable cloth mask with a pocket for receiving a scent releasing layer is disclosed herein as illustrated in FIG. 1. The mask of FIG. 1 shows a scent releasing layer (1) inside of pocket (2) which is sewn on 3 sides to the reusable cloth mask with an open side of the pocket as indicated by the arrow (3). While the non-limiting embodiment of FIG. 1 shows the pocket on the outside of the mask, the pocket for containment of a scent releasing layer may be positioned on the inside surface of the mask.

There exist various considerations with respect to the positioning of the pocket on the inside surface of the reusable cloth mask as opposed to the outside surface of the mask or vice versa. For instance, with respect to considerations relating to the strength of the scent released, the pocket may be positioned on the inner surface for a stronger scent or on the outer surface for a scent of more moderate intensity. Similarly, the pocket may be positioned on the inside of the mask for aesthetic reasons related to hiding the presence of the scented layer or the pocket may be positioned on the outside of the mask to make insertion or replacement of the scented layer more convenient.

The location of the pocket on the reusable cloth mask, whether on the inner or outer surface of the mask, may also vary based on various considerations. In one aspect, positioning of the pocket along a central seam of a reusable mask may provide for manufacturing efficiencies where the central seam not only holds the overall structure of the mask together, but the central seam may also attach one side of the pocket to the mask with a single seam.

The location of the pocket on the reusable cloth mask, may vary based on aesthetic reasons and/or reasons of convenience, such as ease with which a scent releasing strip may be inserted. Thus, in various aspects the location of the pocket can vary from closer to the center of the front or back of the mask to further to the left or right side of the front or back of the mask based on design choice.

In various aspects, the shape of the pocket of the reusable cloth mask can also vary based on design choice and thus, be in any shape of choice. In one aspect, the pocket is in the shape of a quadrilateral which can accommodate a rectangular scent releasing layer. In a further aspect, the pocket is in the shape of a rectangle.

In various aspects, the opening of the pocket may be at the top of the pocket or on a lateral side of the pocket. In one aspect, if the pocket is a rectangular pocket, the opening can be on the side of the pocket furthest from the vertical midline of the mask.

In various aspects, the opening of the pocket may be reversibly closeable with a closure means. Closure of the pocket may be provided by any appropriate means for retaining the scent releasing layer within the pocket. In one aspect, the closure of the pocket may be a strip of hook or loop material on the inside of the pocket, optionally located along the opening, and a companion strip of hook or loop material on the surface of the reusable cloth mask covered by the pocket, optionally located along the opening. In one aspect, the closure may be a flap of material which can cover the opening of the pocket and be fasten closed, for example with hook/loop material or a button.

In various aspects, the reusable cloth mask can be constructed of any material though which a wearer can breathe. In preferred aspects, the material of the reusable cloth mask provides the wearer some level of protection from inhalation of pathogenic microorganisms. In preferred aspects, the material of the reusable cloth mask provides individuals in proximity of the wearer some level of protection from inhalation of pathogenic microorganisms emitted by the nose and/or mouth of the wearer. In various embodiments, the reusable cloth mask is made of a natural cloth material, a synthetic cloth material, or some combination of natural and synthetic materials. In a preferred aspect, the reusable cloth mask is made from cotton.

In various aspects, the pocket is of the same material(s) as the reusable cloth mask or is made of different material(s) as the reusable cloth mask.

In various aspects, the reusable cloth mask comprises one or more antimicrobial compound on the surface of the mask, embedded in the mask, or both on the surface of the mask and embedded in the mask. The one or more antimicrobial compound may be a compound that is resistant to being washed off the reusable cloth mask.

In various aspects, the reusable cloth mask is packaged together with one or more scent releasing layer.

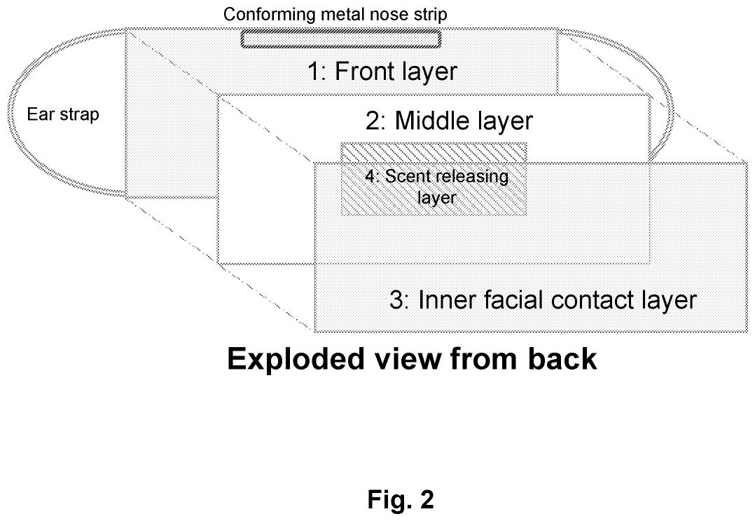

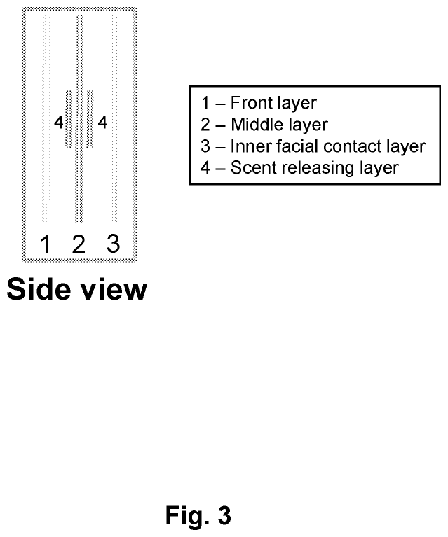

In a further aspect, a disposable mask having an internal scent releasing layer is disclosed herein as illustrated in FIGS. 2 and 3. The mask of FIG. 2 shows an exploded view from back of a disposable mask showing 3 layers for covering the nose and mouth of a wearer including a front layer (1), a middle layer (2), an inner facial contact layer (3). The view further shows a scent releasing layer (4) between the front layer (1) and the inner facial contact layer (3). A side view of the same disposable mask as in FIG. 2 is shown in FIG. 3 showing 3 layers for covering the nose and mouth of a wearer including a front layer (1), a middle layer (2), an inner facial contact layer (3). The side view further shows a scent releasing layer (4) between the front layer (1) and the inner facial contact layer (3). While the side view shows a scent releasing layer (4) on both sides of the middle layer, in some aspects, the scent releasing layer (4) appears in only one of the two illustrated positions.

In various aspects, each individual layer of the disposable mask having an internal scent releasing layer can be constructed of any material through which a wearer can breathe. In certain aspects, the middle layer of the disposable mask can provide for filtering of air inhaled and/or exhaled by the wearer. In preferred aspects, the one or more layers of the disposable mask are constructed with material which provides the wearer some level of protection from inhalation of pathogenic microorganisms. In preferred aspects, the one or more layers of the disposable mask are constructed with material that provides individuals in proximity of the wearer some level of protection from inhalation of pathogenic microorganisms emitted by the nose and/or mouth of the wearer. In various embodiments, the disposable mask comprises a melt-blown polymer. In certain aspects, the disposable mask is made of polypropylene.

In certain aspects the scent releasing layer is positioned between the middle layer and the inner facial contact layer of the mask. In certain aspects, the scent releasing layer is positioned between the middle layer and the outer layer.

In certain aspects, the wherein the scent releasing layer comprises a water soluble polymer selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. In a preferred aspect, the scent releasing laver comprises pullulan.

The scent released from the scent releasing layer can originate from or be provided in the form of an essential oil. In various aspects, the scent releasing layer comprises a compound selected from the group consisting of thymol, methyl salicylate, eucalyptol, menthol and any combination thereof. In certain aspects, the scent is released by the scent releasing layer by activation of essential oils in the scent releasing layer by water vapor in the breath of the mask wearer.

The size of the individual layers of the mask can vary. Is various aspects, all of the front layer, middle layer and inner facial contact layer are flat and cover the nose and mouth of the wearer. In certain aspects, the front layer, middle layer and inner facial contact are rectangular in shape. In a preferred aspect, the front layer, middle layer and inner facial contact are of approximately equal surface area.

The size and positioning of the scent releasing layer can vary. In certain aspects, the scent releasing layer is flat and layered on the proximal surface of the middle layer, e.g. between the middle layer and the inner facial contact layer. In certain aspects, the scent releasing layer is flat and layered on the distal surface of the middle layer, e.g. between the middle layer and the outer layer. The size and shape of the scent releasing layer can vary. In certain aspects, the scent releasing layer is sized to be equal in size to the front layer, middle layer and inner facial contact. In various aspects, the scent releasing layer is smaller in size than the front layer, middle layer and inner facial contact. For example, the front layer, middle layer and inner facial contact can be of equal size and the scent releasing layer can be 90%, 80%, 70%, 60%, 50%, 40%, 30%, 20% or 10% the size, by surface area, of the front layer, middle layer and inner facial contact.

In certain aspects, the scent of the scent releasing layer is designed to expire after a predetermined period of time to indicate that the useful life of the mask has expired.

In various aspects, the disposable mask having an internal scent releasing layer comprises one or more antimicrobial compound on the surface of the mask, embedded in one or more layers of the mask, or both on the surface of the mask and embedded in one or more layers of the mask.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

[1] U.S. Patent Application No. 2019/0320740
[2] U.S. Pat. No. 6,923,981
[3] U.S. Pat. No. 5,538,013
[4] U.S. Patent Application No. 2020/0261684
[5] U.S. Patent Application No. 2012/0144556
[6] U.S. Patent Application No. 2012/0090616

1. A disposable face mask, comprising: an inner facial contact layer, a middle layer and an outer layer, wherein the inner facial contact layer, middle layer and outer layer are each configured to cover the nose and mouth of a user, wherein the mask comprises at least one strap to support the mask on the face of the user, further wherein the mask comprises a scent releasing layer positioned between the inner facial contact layer and the outer layer.

2. The disposable face mask of claim 1, wherein the middle layer provides for filtering of air inhaled and/or exhaled by the user.

3. The disposable face mask of claim 1, wherein the scent releasing layer is positioned between the middle layer and the inner facial contact layer.

4. The disposable face mask of claim 1, wherein the scent releasing layer is positioned between the middle layer and the outer layer.

5. The disposable face mask of claim 1, wherein the scent releasing layer comprises a water soluble polymer selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

6. The disposable face mask of claim 5, wherein the water soluble polymer is pullulan.

7. The disposable face mask of claim 1, wherein the scent releasing layer comprises essential oils.

8. The disposable face mask of claim 1, wherein the scent releasing layer comprises a compound selected from the group consisting of thymol, methyl salicylate, eucalyptol, menthol and any combination thereof.

9. The disposable face mask of claim 1, wherein the facial contact layer, middle layer and outer layer each comprises a melt-blown polymer.

10. The disposable face mask of claim 9, wherein the melt-blown polymer is polypropylene.

11. The disposable face mask of claim 3, wherein the scent releasing layer is layered on the proximal surface of the middle layer.

12. The disposable face mask of claim 4, wherein the scent releasing layer is layered on the distal surface of the middle layer.

13. The disposable face mask of claim 1, wherein the scent of the scent releasing layer is designed to expire after a predetermined period of time to indicate that the useful life of the mask has expired.

14. A reusable cloth face mask, comprising an inner facial contact surface and an outer surface, wherein the inner facial contact surface and the outer surface are configured to cover the nose and mouth of a user, wherein the mask comprises at least one strap to support the mask on the face of the user, further wherein either the inner facial contact surface or the outer surface of the mask comprises a pocket which is smaller in surface area than either the inner facial contact surface or the outer surface, wherein the pocket is attached to the inner or outer surface of the mask on all but one side, wherein the unattached side of the pocket provides an opening allowing access to a cavity within the pocket.

15. The reusable cloth face mask of claim 14, wherein the pocket is stitched on to the inner facial contact surface.

16. The reusable cloth face mask of claim 14, wherein the pocket is stitched on to the outer surface.

17. The reusable cloth face mask of claim 14, wherein the pocket is in the shape of a quadrilateral.

18. The reusable cloth face mask of claim 14, wherein the mask comprises a central vertical stitched seam, wherein one side of the pocket is positioned such that the vertical seam sews the one side of the pocket to the inner or outer surface of the mask.

19. The reusable cloth face mask of claim 14, wherein the opening of the pocket is located opposite the side of the pocket sewn into the central vertical seam.

20. The reusable cloth face mask of claim 14, wherein the pocket is made of cloth.

21. The reusable cloth face mask of claim 14, wherein the inner facial surface and/or the outer surface comprises an antimicrobial compound.

22. The reusable cloth face mask of claim 14, wherein the face mask is packaged in the same packaging as a scent releasing layer which is sized to fit in the pocket.

23. The reusable cloth face mask of claim 14, wherein the pocket further comprises a means for closing access to the cavity within the pocket.

24. A method of making a disposable mask which indicates an observable expiration of useful life by:
(1) making a disposable face mask, comprising: an inner facial contact layer, a middle layer and an outer layer, wherein the inner facial contact layer, middle layer and outer layer are each configured to cover the nose and mouth of a user, wherein the mask comprises at least one strap to support the mask on the face of the user, further wherein the mask comprises a scent releasing layer positioned between the inner facial contact layer and the outer layer; and
(2) designing the scent releasing layer such that the scent of the scent releasing layer expires after a predetermined period of time to indicate to the user that the useful life of the mask has expired.
